(19) United States
(12) Patent Application Publication
(10) Pub. No.: US 2026/0224201 A1
(43) Pub. Date: Aug. 6, 2026

(54) ULTRASONIC DIAGNOSTIC DEVICE AND ULTRASONIC DIAGNOSTIC METHOD

(71) Applicant: SAMSUNG MEDISON CO., LTD., Hongcheon-gun, Gangwon-do (KR)

(72) Inventors: KWANG JU Lee, Hongcheon-gun, Gangwon-do (KR); Gae Young Cho, Hongcheon-gun, Gangwon-do (KR)

(73) Assignee: SAMSUNG MEDISON CO., LTD., Hongcheon-gun, Gangwon-do (KR)

(21) Appl. No.: 19/149,507

(22) PCT Filed: May 10, 2023
(86) PCT No.: PCT/KR2023/006385

§ 371 Date: Jul. 18, 2025

(30) Foreign Application Priority Data
Feb. 9, 2023 (KR) 10-2023-0017288

(51) Int. Cl. A61B 8/00

(52) U.S. Cl. CPC A61B 8/4461; A61B 8/4405; A61B 8/4483; A61B 8/5207

(57) ABSTRACT

An ultrasonic diagnostic device according to an embodiment of the present disclosure includes a transceiver configured to transmit an ultrasound signal to an object and receive a reflected ultrasound signal reflected from the object, a data acquisition unit configured to operate to obtain frame data corresponding to each of a plurality of frames generated based on the transceived signal, and an image compounding unit configured to form an ultrasound spatial compound image by performing spatial compounding on the plurality of pieces of frame data, wherein transmission (Tx) includes Tx steered to the right (right steer Tx) and Tx steered to the left (left steer Tx) only.

### TECHNICAL FIELD

The present disclosure relates to an ultrasonic diagnostic device, an ultrasonic diagnostic method, and an image compounding method for improving the quality of an ultrasound image.

### BACKGROUND ART

An ultrasound image is obtained by transmitting high frequency sound waves from the surface of the human body to the interior of the human body and then imaging the sound waves reflected from the interior. An ultrasonic diagnostic device is a device for obtaining an image of an internal portion of an object by irradiating the object with an ultrasound signal generated from a transducer of a probe and receiving information about an echo signal reflected from the object. The ultrasonic diagnostic device has the advantage of being more stable than a diagnosis device using X-rays and being able to display images in real time. Thus, ultrasonic diagnostic devices have been widely used together with other imaging diagnosis devices.

An image compounding method to improve the quality of an ultrasound image may include a method to perform spatial compounding on a plurality of frames to generate an image. Spatial compounding technology is a technology whereby ultrasound multiple times are transmitted in different directions, a plurality of frames are generated using reception signals reflected from an object, and the plurality of frames are combined to obtain a final image.

However, a tissue image acquired through ultrasound does not appear with uniform brightness, but rather shows many small speckle spots, which are referred to as speckle patterns.

A speckle pattern is a phenomenon caused by scattering in an ultrasound image and occurs because most of scatters in the human body tissue are small in size, densely distributed, and unevenly distributed. A speckle pattern occurs because, when ultrasound is scattered from tissues including scatters in the human body, scattered waves reflected from the respective scatters interfere with each other, causing constructive interference or destructive interference.

As a speckle pattern varies depending on the direction in which the ultrasound is transmitted, the occurrence of the speckle pattern may be reduced by using the spatial compounding technology to transmit ultrasound in different directions and generate a plurality of frames.

However, in the case of the spatial compounding technology, while the quality of an image may be improved, a plurality of frames are needed to generate one image frame, and thus, a frame rate is reduced and artifacts occur when an object or a probe moves while acquiring the image.

Thus, there is a need to develop a technology to minimize frame rate degradation and artifacts caused by movement while improving the quality of an image.

### DISCLOSURE OF INVENTION

### Technical Problem

The present disclosure is directed to providing an ultrasonic diagnostic device and method to increase an imaging speed, increase sharpness, and minimizes the occurrence of an image dragging phenomenon by minimizing the number of Tx steer frames used in an ultrasound spatial compounding process.

### Solution to Problem

An ultrasonic diagnostic device according to the present disclosure includes a transceiver configured to transmit an ultrasound signal to an object and receive a reflected ultrasound signal reflected from the object, a data acquisition unit configured to operate to obtain frame data corresponding to each of a plurality of frames generated based on the transceived signal, and an image compounding unit configured to form an ultrasound spatial compound image by performing spatial compounding on the plurality of pieces of frame data, wherein transmission Tx includes Tx steered to right (right steer Tx) and Tx steered to left (left steer Tx) only.

In detail, the transmission Tx may not include a frame by Tx that is not steered (non-steer frame).

In detail, scan lines of the transmission Tx may be all steered.

In detail, a plurality of frames (Tx steer frames) according to the transmission Tx may include a frame formed by the Tx steered to the right (Tx right frame) and a frame formed by the Tx steered to the left (Tx left frame).

In detail, frames according to the transmission Tx (Tx steer frame) may be even-numbered.

In detail, the number of reception Rx scan lines having different reception Rx settings per transmission Tx may be two or more.

In detail, the transmission Tx may include a plurality of transmissions Tx, and two or more reception Rx scan lines may be obtained from each transmission Tx.

In detail, the two or more reception Rx scan lines according to the same transmission Tx may have the same focusing delay.

In detail, a plurality of reception apertures (Rx apertures) may differ in at least one of a size, a position, and an apodization window.

In detail, weight centers of the plurality of reception apertures (Rx apertures) may move for each depth.

In detail, the plurality of reception apertures (Rx apertures) may move independently.

In detail, the data acquisition unit may obtain one piece of frame data by using the plurality of reception Rx scan lines generated from the same transmission Tx.

In detail, compounding signals of the plurality of reception Rx scan lines may be performed by applying respective weights to signals of the plurality of reception Rx scan lines.

In detail, the weight may differ depending on a depth.

In detail, a scan line of the transmission Tx that forms the frame formed by the Tx steered to the right (Tx right frame) and a scan line of the transmission Tx that forms the frame formed by the Tx steered to the left (Tx left frame) may each obtain two or more reception Rx scan lines.

In detail, the weight centers of reception apertures (Rx apertures) of a plurality of reception Rx scan lines may be different from each other.

An ultrasound diagnostic method according to the present disclosure includes transmitting, by a transceiver, an ultrasound signal to an object and receiving, by the transceiver, a reflected ultrasound signal reflected from the object, operating a data acquisition unit to obtain frame data corresponding to each of a plurality of frames generated based on the transceived signal, and forming, by an image compounding unit, an ultrasound spatial compound image by performing spatial compounding on a plurality of pieces of frame data, wherein transmission Tx may include Tx steered to right (right steer Tx) and Tx steered to left (left steer Tx) only.

In detail, a plurality of frames (Tx steer frame) according to the transmission Tx may include the frame formed by Tx steered to the right (Tx right frame) and the frame formed by the Tx steered to the left (Tx left frame).

In detail, obtaining two or more reception Rx scan lines having different reception Rx settings per transmission Tx may be included.

In detail, the two or more reception Rx scan lines according to the same transmission Tx may have the same focusing delay.

In detail, a plurality of reception apertures (Rx apertures) may differ in at least one of a size, a position, and an apodization window.

In detail, weight centers of a plurality of reception apertures (Rx apertures) may move for each depth.

In detail, a plurality of reception apertures (Rx apertures) may move independently.

In detail, obtaining, by the data acquisition unit, one piece of frame data by using N reception Rx scan lines generated from the same transmission Tx may be included.

In detail, compounding signals of the plurality of reception Rx scan lines by applying respective weights to signals of the plurality of reception Rx scan lines may be included.

In detail, the weight may differ depending on a depth.

### Advantageous Effects of Invention

According to the ultrasonic diagnostic device and method according to the present disclosure, in an ultrasound spatial compounding process, an imaging speed and sharpness may be increased, occurrence of an image dragging phenomenon may be minimized, and a speckle reduction effect may be increased.

The effects of the present disclosure are not limited to the effects described above, and other various effects that are not mentioned herein would be clearly understood by a person skilled in the art from the description of the disclosure and the accompanying drawings.

### BRIEF DESCRIPTION OF DRAWINGS

FIG. 1 is a block diagram illustrating a configuration of an ultrasonic diagnostic device 100 according to any one embodiment of the present disclosure.

FIG. 2 is a block diagram illustrating the configuration of the ultrasonic diagnostic device 100 according to any one embodiment of the present disclosure.

FIG. 3 is a block diagram illustrating the configuration of the ultrasonic diagnostic device 100 according to any one embodiment of the present disclosure.

(a) to (c) of FIG. 4 are perspective views of an ultrasonic diagnostic device 200 according to at least one embodiment of the present disclosure.

(a) to (c) of FIG. 5 are perspective views of an ultrasonic diagnostic device 500 according to at least one embodiment of the present disclosure.

FIG. 6 is a diagram for explaining an ultrasound spatial compound imaging method according to the related art.

FIGS. 7A to 7D are diagrams for explaining a transceiving method in the ultrasound spatial compound imaging method according to the related art.

FIGS. 8A to 8D are diagrams for explaining a transceiving method of the ultrasound spatial compound imaging method, in an ultrasonic diagnostic device 700 according to an embodiment of the present disclosure.

FIGS. 9A and 9B are diagrams for comparing the ultrasound transceiving method according to the related art and the ultrasound transceiving method according to an embodiment of the present disclosure, in the ultrasound spatial compound imaging method.

FIG. 10 is a diagram for explaining a position of a reception aperture in the ultrasound spatial compound imaging method according to an embodiment of the present disclosure.

FIG. 11 is a diagram for explaining a position of a reception aperture in an ultrasound spatial compound imaging method according to another embodiment of the present disclosure.

FIG. 12 is a diagram for explaining a position of a reception aperture position in an ultrasound spatial compound imaging method according to another embodiment of the present disclosure.

FIG. 13 is a diagram for explaining an ultrasound spatial compound imaging method according to an embodiment of the present disclosure.

FIG. 14 is a diagram for explaining a case in which Rx apodization windows are different from one another, in an ultrasound spatial compound imaging method according to another embodiment of the present disclosure.

FIG. 15 is a diagram for explaining effects of the ultrasound spatial compound imaging method of the present disclosure.

FIG. 16 is a diagram for explaining effects of the ultrasound spatial compound imaging method according to the related art.

FIG. 17 is a diagram for explaining effects of the ultrasound spatial compound imaging method according to the present disclosure.

### MODE FOR THE INVENTION

The present specification describes the principle of the disclosure and discloses embodiments to clarify the scope of rights of the present disclosure and enable one skilled in the art to which the present disclosure pertains to work the present disclosure. The disclosed embodiments may be implemented in various forms.

Throughout the disclosure, when a component is “connected” to another component, the component is connected to the other component not only directly, but also indirectly, and the indirect connection includes a connection through a wireless communication network.

Furthermore, the terms used in the present specification are merely used to describe particular embodiments, and are not intended to limit the disclosure. As used herein, the singular forms “a,” “an,” and “the” are intended to include the plural forms as well, unless the context clearly indicates otherwise. In the present specification, it is to be understood that terms such as “including” or “having,” etc., are intended to indicate the existence of the features, numbers, steps, actions, components, parts, or combinations thereof disclosed in the present specification, and are not intended to preclude the possibility that one or more other features, numbers, steps, actions, components, parts, or combinations thereof may exist or may be added.

Furthermore, in the present specification, while such terms including an ordinal number, such as “first,” “second,” etc., may be used to describe various components, such components must not be limited to the above terms. The above terms are used only to distinguish one element from another. For example, without departing from the right scope of the disclosure, a first component may be referred to as a second component, and vice versa.

Furthermore, the term “ . . . portions”, “ . . . units”, “ . . . blocks”, “ . . . members”, or “ . . . modules” denote units that process at least one function or operation. For example, the terms may denote at least one of processes processed by at least one hardware, such as a field-programmable gate array (FPGA)/an application specific integrated circuit (ASIC), etc., at least one software stored in a memory, or a processor.

A sign attached to each operation is used to identify each operation. These signs do not indicate an order of respective operations, and the respective operations may be implemented differently from the specified order unless a specific order is clearly stated in the context.

Furthermore, in the present specification, an image may include a medical image obtained by a medical imaging apparatus, such as a magnetic resonance imaging (MRI) device, a computed tomography (CT), an ultrasound imaging device, or an X-ray imaging device, and may provide or control ultrasound images and medical images of modalities other than ultrasound.

Furthermore, in the present specification, the term “object” refers to a subject to be imaged and may include a human, an animal, or a part thereof. For example, the object may include a part of a body (e.g., an organ or a body structure) or a phantom.

Throughout the specification, the term “ultrasound image” refers to an image of the object that is generated or processed based on ultrasound signals transmitted to and reflected from the object.

In the following description, an embodiment according to the present disclosure is described in detail with reference to the accompanying drawings.

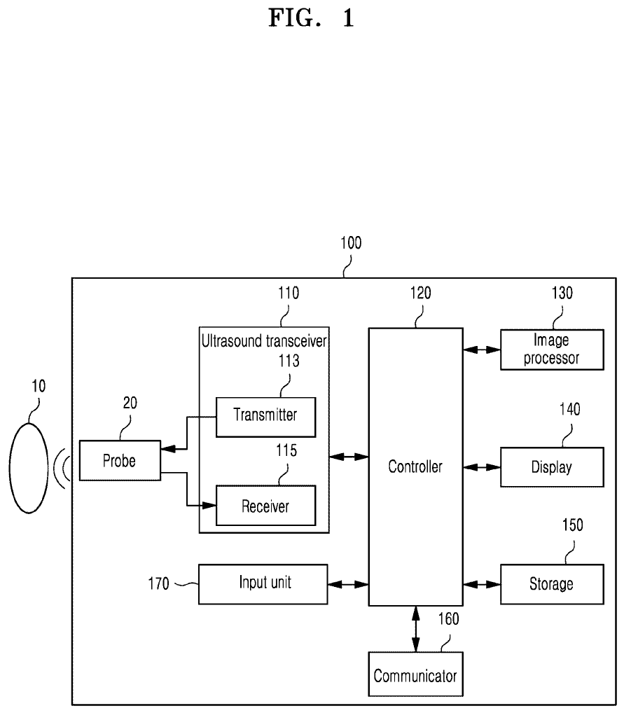

FIG. 1 is a block diagram illustrating a configuration of an ultrasonic diagnostic device 100 according to any one embodiment of the present disclosure.

The ultrasonic diagnostic device 100 according to an embodiment may include a probe 20, an ultrasound transceiver 110, a controller 120, an image processor 130, a display 140, a storage 150, a communicator 160, and an input unit 170.

The ultrasonic diagnostic device 100 may be implemented not only as a cart type, but also as a portable type. Examples of a portable ultrasonic diagnostic device may include a probe, a smart phone, a laptop computer, a personal digital assistants (PDA), or a tablet personal computer (PC) each including an application, but the disclosure is not limited thereto.

The probe 20 may include a plurality of transducers. The plurality of transducers may transmit ultrasound signals to an object 10 according to transmission signals applied by a transmitter 113. The plurality of transducers may receive the ultrasound signals reflected from the object 10 and form reception signals. Furthermore, the probe 20 may be implemented in an integrated from with the ultrasonic diagnostic device 100 or in a separate from connected to the ultrasonic diagnostic device 100 in a wired or wireless manner. Furthermore, the ultrasonic diagnostic device 100 may be provided with one or the plurality of probes 20 according to the implementation form.

The controller 120 controls the transmitter 113 to form a transmission signal applied to each of the plurality of transducers, considering positions and focus points of the plurality of transducers included in the probe 20.

The controller 120 controls a receiver 115 to generate ultrasound data by performing analog-digital conversion on the reception signals received from the probe 20 and summing the digitally converted reception signals considering the positions and the focus points of the plurality of transducers.

The image processor 130 generates an ultrasound image by using the ultrasound data generated by the receiver 115.

The ultrasound image may be represented not only by an ultrasound image in a gray scale by scanning an object according to A mode (amplitude mode), B mode (brightness mode), and M mode (motion mode), but also by a Doppler image showing a movement of the object.

The A mode, which is the most basic form of an ultrasound image display method, is a method of indicating the strength of reflected sound by an amplitude size on a time (distance) axis. The A mode is advantageous for distance measurement because the amplitude is high when the reflected sound is strong and low when the reflected sound is weak. However, the A mode is rarely used at present because an image changes even when the direction of a probe is slightly different.

The M mode is a modified form of the A mode that displays the distance of a moving reflector as a time change. The M mode designates a region of interest (ROI) within a two-dimensional (2D) image as an M line and displays changes in the region over time. The M mode is mainly used to observe heart valves and is also able to record heart sounds in fetuses, but the mode is recently being largely replaced by the Doppler method.

The B mode is a method that displays reflected sound as brightness of dots, and is currently used in most ultrasound diagnosis equipment. The brightness of each dot is proportional to the amplitude of a reflected signal. Recently, the B mode provides 256 or more brightness levels and is also a mode that displays movements of organs as a real-time image. A mode called a 2D mode refers to a brightness (B) mode that displays a cross-sectional image of an object in real time on a screen in black and white shades, which is the most used mode

In addition, a Doppler mode is generally a mode that measures blood flow by detecting the flow of red blood cells in a blood vessel. The mode uses the principle that wavelength becomes shorter when the red blood cells approach a probe and longer when the red blood cells move away. Depending on the method of displaying blood flow, there are color Doppler, pulse wave Doppler (PW), and continuous wave Doppler (CW). Doppler images may include blood flow Doppler images (also called color Doppler images) that show the flow of blood, tissue Doppler images that show the movements of tissues, and spectral Doppler images that display the movement speed of an object as a waveform.

In addition, as a composite mode, there are a mode that simultaneously applies two or three modes to one image and displays other modes together based on 2D, and a three-dimensional (3D) mode that displays a 3D stereoscopic image.

In a B mode processing process, B mode components are extracted from the ultrasound data and processed, and in an image generation process, an ultrasound image in which the signal strength is expressed as brightness may be generated based on the B mode components extracted in the B mode processing process. In a Doppler processing process, Doppler components are extracted from the ultrasound data, and in the image generation process, a Doppler image that expresses the movement of the object 10 in color or waveform may be generated based on the extracted Doppler components.

In the image generation process, a 2D ultrasound image or 3D image may be generated for the object, and an elastic image may be generated which images a degree of deformation of the object 10 according to pressure. Furthermore, various pieces of additional information may be expressed on an ultrasound image by text or graphics. The generated ultrasound image may be stored in a memory.

In the process of measuring an object within an ultrasound image, a measurement tool for object measurement may be determined, and one of a plurality of measurement tools may be selected based on a user's input.

For example, a measurement tool selection menu to select one of a plurality of measurement tools may be provided, and the measurement tool selection menu may be displayed on one screen together with an ultrasound image. Furthermore, the measurement tool selection menu may be displayed on a separate screen different from a touch screen on which the ultrasound image is displayed.

Furthermore, one of a plurality of measurement tools may be determined based on a user's input to select one of a plurality of measurement items to be measured. The measurement items may include length, width, or angle, but the disclosure is not limited thereto.

On the reception of the user's input to select one of the measurement items, a predetermined measurement tool corresponding to the selected measurement item may be determined.

The display 140 may display the generated ultrasound image and various pieces of information processed in the ultrasonic diagnostic device 100. The ultrasonic diagnostic device 100 may include one or a plurality of displays as the display 140 according to the implementation form. Furthermore, the display 140 may be coupled to a touch panel to be implemented as a touch screen.

The controller 120 may control the overall operation of the ultrasonic diagnostic device 100 and a signal flow between the internal components of the ultrasonic diagnostic device 100. The controller 120 may include a memory for storing a program or data to perform the function of the ultrasonic diagnostic device 100, and a processor for processing the program or data. Furthermore, the controller 120 may receive a control signal from the input unit 170 or an external device and may control the operation of the ultrasonic diagnostic device 100.

The ultrasonic diagnostic device 100 may include the communicator 160, and may be connected to an external device (e.g., a server, a medical device, a portable device (a smartphone, a tablet PC, a wearable device, etc.)) through the communicator 160.

The communicator 160 may include one or more components that enable a communication with the external device, and include at least one of, for example, a short-range communication module or a wired communication module and wireless communication module.

It is possible for the communicator 160 to receive a control signal and data from the external device and transmit a control signal and data to the external device, and to transmit the received control signal to the controller 120 so that the controller 120 controls the ultrasonic diagnostic device 100 according to the received control signal.

Alternatively, by transmitting a control signal to the external device through the communicator 160, it is possible for the controller 120 to control the external device according to the control signal of the controller.

For example, the external device may process data of the external device according to the control signal of the controller received through the communicator.

As a program (artificial intelligence, etc.) for controlling the ultrasonic diagnostic device 100 may be installed on the external device, the program may include instructions to perform part or the whole of the operation of the controller 120.

The program may be previously installed on the external device, or a user of the external device may download the program from a server that provides an application and install the downloaded program. The server that provides the application may include a recording medium storing the corresponding program.

Furthermore, in a system including a server and a client device, the program product may include a storage medium of the server or a storage medium of the client device. Alternatively, when there is a third device (a smartphone, a tablet PC, a wearable device, etc.) that is communicatively connected to the server or the client device, the program product may include a storage medium of a third device. Alternatively, the program may include a S/W program transmitted from the server to the client device or the third device or from the third device to the client device.

In this case, as one of the server, the client device, and the third device executes the program, the method according to the disclosed embodiments may be performed. Alternatively, as two or more of the server, the client device, and the third device execute the program, the method according to the disclosed embodiments may be performed in a distributed manner.

For example, as the server (e.g., a cloud server, an artificial intelligence server, etc.) executes the program stored in the server, the client device communicatively connected to the server may be controlled to perform the method according to the disclosed embodiments.

The storage 150 may store various pieces of data or programs for driving and controlling the ultrasonic diagnostic device 100, ultrasound data that is input/output, and the obtained ultrasound image.

The input unit 170 may receive a user's input to control the ultrasonic diagnostic device 100. For example, the user's input may include an input for manipulating a button, a key pad, a mouse, a trackball, a jog switch, or a knop, an input for touching a touch pad or a touch screen, a voice input, a motion input, or a biometric information input (e.g., iris recognition, fingerprint recognition, etc.), but the disclosure is not limited thereto.

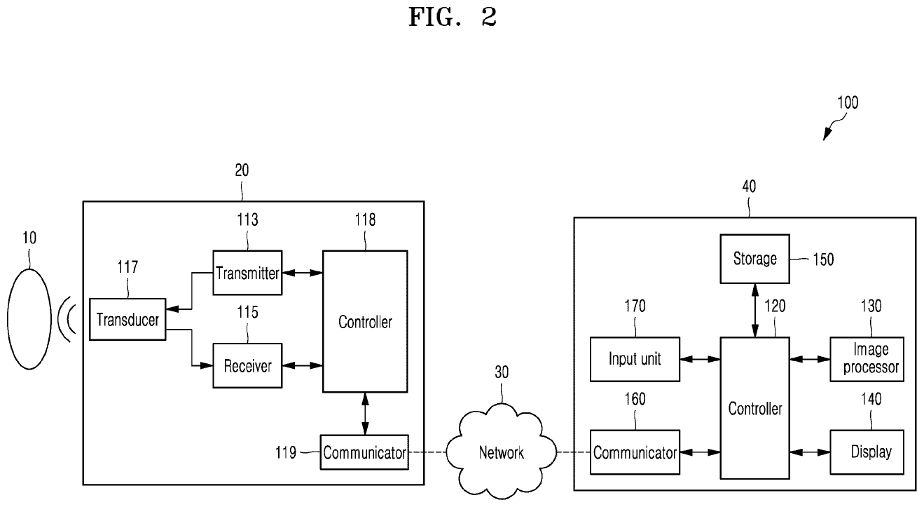

FIG. 2 is a block diagram illustrating the configuration of the ultrasonic diagnostic device 100 according to any one embodiment of the present disclosure.

Referring to FIG. 2, the ultrasonic diagnostic device 100 may include a wireless probe 20 and an ultrasound system 40.

The wireless probe 20 may include the transmitter 113, a transducer 117, the receiver 115, a controller 118, and a communicator 119. In FIG. 2, the wireless probe 20 is illustrated as including both of the transmitter 113 and the receiver 115, but according to the implementation form, the wireless probe 20 may include only part of the configurations of the transmitter 113 and the receiver 115, and a part of the configurations of the transmitter 113 and the receiver 115 may be included in the ultrasound system 40. Alternatively, the wireless probe 20 may further include the image processor 130.

The transducer 117 may include a plurality of transducers. The plurality of transducers may transmit ultrasound signals to the object 10 according to the transmission signal applied from the transmitter 113. The plurality of transducers may form reception signals by receiving the ultrasound signals reflected from the object 10.

The controller 118 controls the transmitter 113 to form a transmission signal applied to each of the plurality of transducers, considering the positions and the focus points of the plurality of transducers.

The controller 118 controls the receiver 115 to generate ultrasound data by performing analog-digital conversion on the reception signal received from the probe 20 and summing the digitally converted reception signals considering the positions and the focus points of the plurality of transducers. Alternatively, when the wireless probe 20 includes the image processor 130, an ultrasound image may be generated by using the generated ultrasound data.

The communicator 119 may wirelessly transmit the generated ultrasound data or ultrasound image to the ultrasound system 40 through a wireless network. Alternatively, the communicator 119 may receive the control signal and data from the ultrasound system 40.

Furthermore, the ultrasonic diagnostic device 100 may be provided with one or more wireless probes 20 according to the implementation form.

The ultrasound system 40 may receive the ultrasound data or ultrasound image from the wireless probe 20. The ultrasound system 40 may include the controller 120, the image processor 130, the display 140, the storage 150, the communicator 160, and the input unit 170.

The image processor 130 generates an ultrasound image by using the ultrasound data received from the wireless probe 20.

The display 140 may display the ultrasound image received from the wireless probe 20, the ultrasound image generated in the ultrasound system 40, and various pieces of information processed in the ultrasonic diagnostic device 100. The ultrasonic diagnostic device 100 may include one or a plurality of displays as the display 140 according to the implementation form. Furthermore, the display 140 may be coupled to a touch panel to be implemented as a touch screen.

The controller 120 may control the overall operation of the ultrasonic diagnostic device 100 and the signal flow between the internal components of the ultrasonic diagnostic device 100. The controller 120 may include a memory for storing a program or data to perform the function of the ultrasonic diagnostic device 100, and a processor for processing the program or data. Furthermore, the controller 120 may receive a control signal from the input unit 170 or the external device and control the operation of the ultrasonic diagnostic device 100.

The ultrasound system 40 may include the communicator 160, and may be connected to an external device (e.g., a server, a medical device, a portable device (a smartphone, a tablet PC, a wearable device, etc.)) through the communicator 160.

The communicator 160 may include one or more components that enable a communication with the external device, and include at least one of, for example, a short-range communication module or a wired communication module and wireless communication module.

It is possible for the communicator 160 to receive a control signal and data from the external device and transmit a control signal and data to the external device, and to transmit the received control signal to the controller 120 so that the controller 120 controls the ultrasonic diagnostic device 100 according to the received control signal.

Alternatively, by transmitting a control signal to the external device through the communicator 160, it is possible for the controller 120 to control the external device according to the control signal of the controller.

For example, the external device may process data of the external device according to the control signal of the controller received through the communicator.

As a program (artificial intelligence, etc.) for controlling the ultrasonic diagnostic device 100 may be installed on the external device, the program may include instructions to perform part or the whole of the operation of the controller 120.

The program may be previously installed on the external device, or a user of the external device may download the program from a server that provides an application and install the downloaded program. The server that provides the application may include a recording medium storing the corresponding program.

Furthermore, in the system including a server and a client device, the program product may include a storage medium of the server or a storage medium of the client device. Alternatively, when there is a third device (a smartphone, a tablet PC, a wearable device, etc.) that is communicatively connected to the server or the client device, a program product may include a storage medium of a third device. Alternatively, the program may include a S/W program transmitted from the server to the client device or the third device or from the third device to the client device.

In this case, as one of the server, the client device, and the third device executes the program, the method according to the disclosed embodiments may be performed. Alternatively, the client device may perform the method according to the disclosed embodiments via the server.

Alternatively, as two or more of the server, the client device, and the third device execute the program, the method according to the disclosed embodiments may be performed in a distributed manner.

For example, as the server (e.g., a cloud server, an artificial intelligence server, etc.) executes the program stored in the server, the client device communicatively connected to the server may be controlled to perform the method according to the disclosed embodiments.

The storage 150 may store various pieces of data or programs for driving and controlling the ultrasonic diagnostic device 100, ultrasound data that is input/output, or ultrasound image.

The input unit 170 may receive the user's input to control the ultrasonic diagnostic device 100. For example, the user's input may include an input for manipulating a button, a key pad, a mouse, a trackball, a jog switch, or a knop, an input for touching a touch pad or a touch screen, a voice input, a motion input, or a biometric information input (e.g., iris recognition, fingerprint recognition, etc.), but the disclosure is not limited thereto.

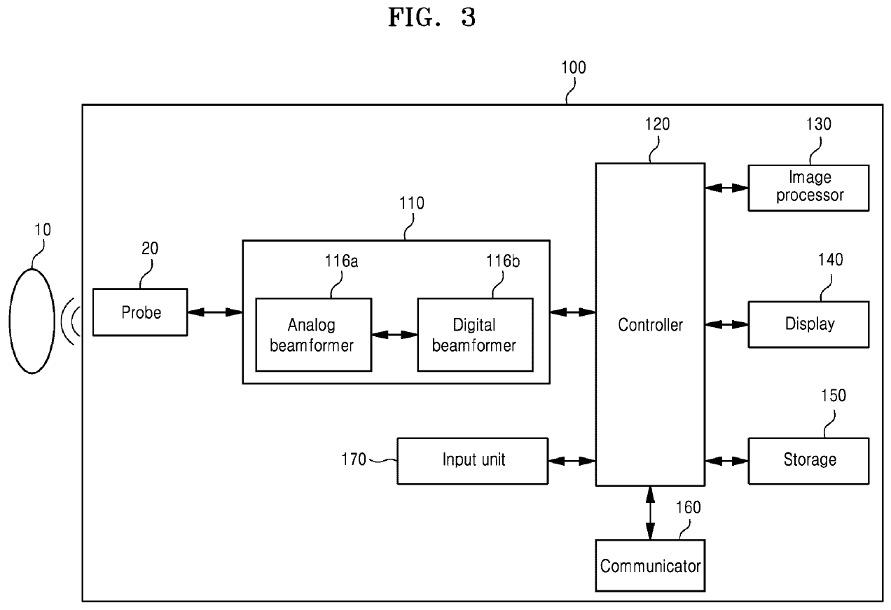

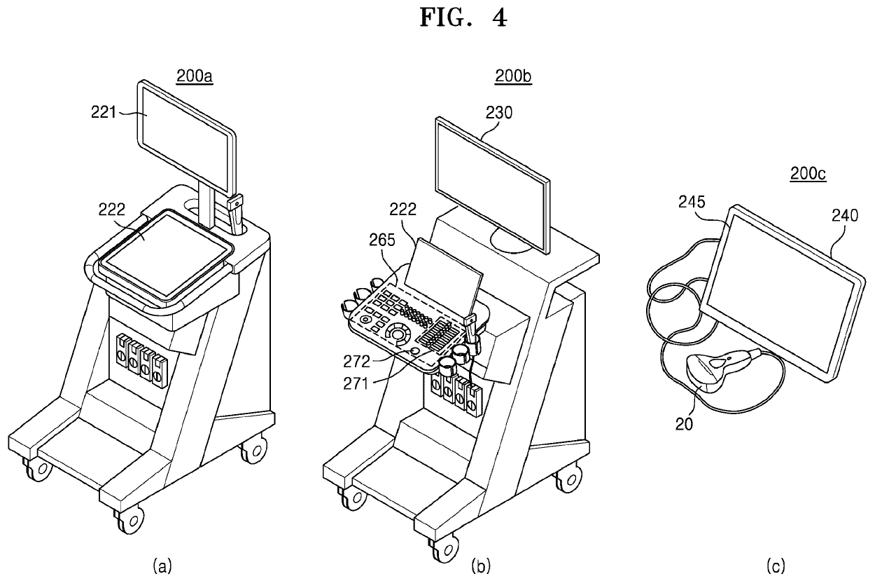

FIG. 3 is a block diagram illustrating the configuration of the ultrasonic diagnostic device 100 according to any one embodiment of the present disclosure.

Referring to FIG. 3, the ultrasonic diagnostic device 100 may include the probe 20, the ultrasound transceiver 110, the controller 120, the image processor 130, the display 140, the input unit 170, the storage 150, and the communicator 160.

The probe 20 according to an embodiment may include a plurality of transducers. The plurality of transducers are arranged two-dimensionally forming a 2D transducer array.

For example, the 2D transducer array may include a sub-array including a plurality of transducers arranged in a first direction, and a plurality of sub-arrays are arranged in a second direction different from the first direction.

Furthermore, the ultrasound transceiver 110 may include an analog beamformer 116a and a digital beamformer 116b. In FIG. 3, the ultrasound transceiver 110 and the probe 20 are illustrated as being separate configurations, but the probe 20 according to an embodiment may include part or the whole of the configurations of the ultrasound transceiver 110, according to the implementation form. For example, the probe 20 may include one or two of the analog beamformer 116a and the digital beamformer 116b.

The controller 120 may calculate a time delay value for digital beamforming for each sub-array with respect to each of each of a plurality of sub-arrays included in the 2D transducer array. Furthermore, the controller 120 may calculate a time delay value for analog beamforming for each of the transducers included in any one of the plurality of sub-arrays.

The controller 120 may control the analog beamformer 116a and the digital beamformer 116b to form a transmission signal applied to each of the plurality of transducers according to the time delay value for analog beamforming and the time delay value for digital beamforming.

Furthermore, the controller 120 may control the analog beamformer 116a to sum the signals received from the plurality of transducers for each sub-array, according to the time delay value for analog beamforming. Furthermore, the controller 120 may control the ultrasound transceiver 110 to perform analog-digital conversion on the summed signal for each sub-array. Furthermore, the controller 120 may control the digital beamformer 116b to generate ultrasound data by summing the digitally converted signals, according to the time delay value for digital beamforming.

The image processor 130 generates an ultrasound image by using the generated ultrasound data.

The display 140 may display the generated ultrasound image and various pieces of information processed in the ultrasonic diagnostic device 100. The ultrasonic diagnostic device 100 may include one or a plurality of displays as the display 140 according to the implementation form. Furthermore, the display 140 may be coupled to a touch panel to be implemented as a touch screen.

The controller 120 may control the overall operation of the ultrasonic diagnostic device 100 and the signal flow between the internal components of the ultrasonic diagnostic device 100. The controller 120 may include a memory for storing a program or data to perform the function of the ultrasonic diagnostic device 100, and a processor for processing the program or data. Furthermore, the controller 120 may receive a control signal from the input unit 170 or the external device and control the operation of the ultrasonic diagnostic device 100.

The ultrasonic diagnostic device 100 may include the communicator 160, and may be connected to an external device (e.g., a server, a medical device, a portable device (a smartphone, a tablet PC, a wearable device, etc.)) through the communicator 160.

The communicator 160 may include one or more components that enable a communication with the external device, and include at least one of, for example, a short-range communication module or a wired communication module and wireless communication module.

It is possible for the communicator 160 to receive a control signal and data from the external device transmit a control signal and data to the external device, and to transmit the received control signal to the controller 120 so that the controller 120 controls the ultrasonic diagnostic device 100 according to the received control signal.

Alternatively, by transmitting a control signal to the external device through the communicator 160, it is possible for the controller 120 to control the external device according to the control signal of the controller.

For example, the external device may process data of the external device according to the control signal of the controller received through the communicator.

As a program (artificial intelligence, etc.) for controlling the ultrasonic diagnostic device 100 may be installed on the external device, the program may include instructions to perform part or the whole of the operation of the controller 120.

The program may be previously installed on the external device, or a user of the external device may download the program from a server that provides an application and install the downloaded program. The server that provides the application may include a recording medium storing the corresponding program.

Furthermore, in the system including a server and a client device, the program product may include a storage medium of the server or a storage medium of the client device. Alternatively, when there is a third device (a smartphone, a tablet PC, a wearable device, etc.) that is communicatively connected to the server or the client device, a program product may include a storage medium of a third device. Alternatively, the program may include a S/W program transmitted from the server to the client device or the third device or from the third device to the client device.

In this case, one of the server, the client device, and the third device may execute the program to perform the method according to the disclosed embodiments. Alternatively, two or more of the server, the client device, and the third device may execute the program to perform the method according to the disclosed embodiments in a distributed manner.

For example, as the server (e.g., a cloud server, an artificial intelligence server, etc.) executes the program stored in the server, the client device communicatively connected to the server may be controlled to perform the method according to the disclosed embodiments.

The storage 150 may store various pieces of data or programs for driving and controlling the ultrasonic diagnostic device 100, ultrasound data that is input/output, or an ultrasound image.

The input unit 170 may receive a user's input to control the ultrasonic diagnostic device 100. For example, the user's input may include an input for manipulating a button, a key pad, a mouse, a trackball, a jog switch, or a knop, an input for touching a touch pad or a touch screen, a voice input, a motion input, or a biometric information input (e.g., iris recognition, fingerprint recognition, etc.), but the disclosure is not limited thereto.

(a) to (c) of FIG. 4 are perspective views of an ultrasonic diagnostic device 200 according to at least one embodiment of the present disclosure.

Referring to (a) and (b) of FIG. 4, ultrasonic diagnostic devices 200a and 200b may include a main display 221 and a sub-display 222. One of the main display 221 and the sub-display 222 may be implemented as a touch screen. The main display 221 and the sub-display 222 may each display ultrasound images or various pieces of information processed in the ultrasonic diagnostic devices 200a and 200b. Furthermore, the main display 221 and the sub-display 222 may each be implemented as a touch screen, and by providing a graphical user interface (GUI), may receive, from a user, data for controlling the ultrasonic diagnostic devices 200a and 200b. For example, the main display 221 may display an ultrasound image, and the sub-display 222 may display a control panel for controlling display of the ultrasound image in the form of a GUI. The sub-display 222 may receive data for controlling display of an image, through the control panel displayed in the form of a GUI. The ultrasonic diagnostic devices 200a and 200b may control display of the ultrasound image displayed on the main display 221, by using the received control data.

Referring to (b) of FIG. 4, the ultrasonic diagnostic device 200b may further include a control panel 265 other than the main display 221 and the sub-display 222. The control panel 265 may include a button, a trackball, a jog switch, or a knob, and may receive from a user data for controlling the ultrasonic diagnostic device 200b. For example, the control panel 265 may include a time gain compensation (TGC) button 271 or a freeze button 272. The TGC button 271 may be a button for setting a TGC value of an ultrasound image for each depth. Furthermore, when detecting an input of the freeze button 272 while scanning an ultrasound image, the ultrasonic diagnostic device 200b may maintain a state in which a frame image at the corresponding time is displayed.

The button, the trackball, the jog switch, or the knob included in the control panel 265 may be provided to the main display 221 or the sub-display 222 in the form of a GUI.

Referring to (c) of FIG. 4, an ultrasonic diagnostic device 200c may be implemented as a portable type. Examples of the ultrasonic diagnostic device 200c of a portable type may include smart phones, laptop computers, PDAs, or tablet PCs, which each include a probe and an application, but the disclosure is not limited thereto.

The ultrasonic diagnostic device 200c may include the probe 20 and a main body 240, and the probe 20 may be connected to one side of the main body 240 in a wired or wireless manner. The main body 240 may include a touch screen 245. The touch screen 245 may display an ultrasound image, various pieces of information processed in the ultrasonic diagnostic device, or GUI.

(a) to (c) of FIG. 5 are perspective views of an ultrasonic diagnostic device 500 according to at least one embodiment of the present disclosure.

Referring to (a) of FIG. 5, an ultrasonic diagnostic device used indoors or the ultrasonic diagnostic device 500 for indoor use may refer to an ultrasonic diagnostic device that is generally used for ultrasound diagnosis and is not portable, and the ultrasonic diagnostic device 500 may be referred to as cart-based equipment. Although it is not necessary for the ultrasonic diagnostic device 500 to be used indoors, for convenience, the ultrasonic diagnostic device 500 is referred to as the ultrasonic diagnostic device 500 for indoor use.

The ultrasonic diagnostic device 500 for indoor use may have a portable docking portion 580 connected to a portable ultrasonic diagnostic device 400, and components of the ultrasonic diagnostic device 500 for indoor use described in the present disclosure, excluding the portable docking portion 580, are generally used components, and thus detailed descriptions thereof are omitted.

As the ultrasonic diagnostic device 500 for indoor use has fewer constraints in terms of size, weight, power consumption, etc., unlike the portable ultrasonic diagnostic device 400, the ultrasonic diagnostic device 500 for indoor may be developed with a variety of diagnostic items and high performance. When the portable ultrasonic diagnostic device 400 is mounted on the ultrasonic diagnostic device 500 for indoor use, the portable ultrasonic diagnostic device 400 may be used as a high performance device. The position where the portable ultrasonic diagnostic device 400 is mounted on the ultrasonic diagnostic device 500 for indoor use is not limited if only the position is where the portable ultrasonic diagnostic device 400 and the ultrasonic diagnostic device 500 for indoor use are simultaneously and easily used, and is not limited by (a) of FIG. 5. Furthermore, the portable ultrasonic diagnostic device 400 may be connected to the ultrasonic diagnostic device 500 for indoor use via a wired connection or may be integrally combined therewith. Referring to (a) and (b) of FIG. 5, the portable ultrasonic diagnostic device 400 in (a) of FIG. 5 may correspond to a portable ultrasonic diagnostic device 201 in (b) of FIG. 5.

The portable ultrasonic diagnostic device 400 may be integrally formed with a probe (not shown) including a plurality of transducer elements. In detail, the portable ultrasonic diagnostic device 400 may refer to a device that is connected to the ultrasonic diagnostic device 500 for indoor use by using a wireless or wired communication method (including a universal serial bus (USB)) and provides a user with an ultrasound image by using received ultrasound image data. In an example, the portable ultrasonic diagnostic device 400 may be a smart device such as a smartphone, on which an application is downloaded and installed.

In detail, the portable ultrasonic diagnostic device 400 may be a device that is connected to the ultrasonic diagnostic device 500 for indoor use by a wired or wireless communication method and provides a user an ultrasound image by using the received ultrasound image data.

In an example, the wireless communication method may include at least one of short-range data communication methods including a 60 GHz (mmWave) wireless short-range communication network (WLAN). The wireless communication method may be a short-range wireless network (Wi-Fi), Bluetooth, Zigbee, Wi-Fi direct (WFD), Infrared Data Association (IrDA), Bluetooth low energy (BLE), near field communication (NFC), wireless broadband Internet (Wibro), shared wireless access protocol (SWAP) for worldwide interoperability for microwave access (WiMAX), wireless gigabit alliance (WiGig), or wireless frequency (RF) communication.

(b) of FIG. 5 shows an example of an ultrasound diagnosis system in which the portable ultrasonic diagnostic device 201 is connected to the cart-based ultrasonic diagnostic device 500.

The cart-based ultrasonic diagnostic device 500 may be connected to the portable ultrasonic diagnostic device 201 by using the wireless communication method described above. In detail, the portable ultrasonic diagnostic device 201 may include at least one wireless communication module (not shown) to perform at least one of the wireless communication methods described above. Furthermore, the portable docking portion 580 of the cart-based ultrasonic diagnostic device 500 may include at least one wireless communication module (not shown) to perform a wireless communication with the portable ultrasonic diagnostic device 201.

In this state, the wireless communication module in the cart-based ultrasonic diagnostic device 500 may be a module for performing communication according to at least one of the wireless communication methods described above.

(c) of FIG. 5 shows an example of an ultrasound diagnosis system in which a portable ultrasonic diagnostic device 202 is connected to the cart-based ultrasonic diagnostic device 500.

The portable ultrasonic diagnostic device 202 may be connected to a probe 301 through a probe port. The portable ultrasonic diagnostic device 202 may generate an ultrasound image and display the generated ultrasound image on a display by using an ultrasound image corresponding to an ultrasound signal received by the probe 301.

The cart-based ultrasonic diagnostic device 500 may be connected to the portable ultrasonic diagnostic device 202 by using the wireless communication method described above. As the connection between the cart-based ultrasonic diagnostic device 500 and the portable ultrasonic diagnostic device 202 through communication corresponds to the connection between the cart-based ultrasonic diagnostic device 500 and the portable ultrasonic diagnostic device 201, a detailed description thereof is omitted.

In the following description, an embodiment of an ultrasound device applicable to at least one of the ultrasonic diagnostic devices described with reference to FIGS. 1 to 3 is described.

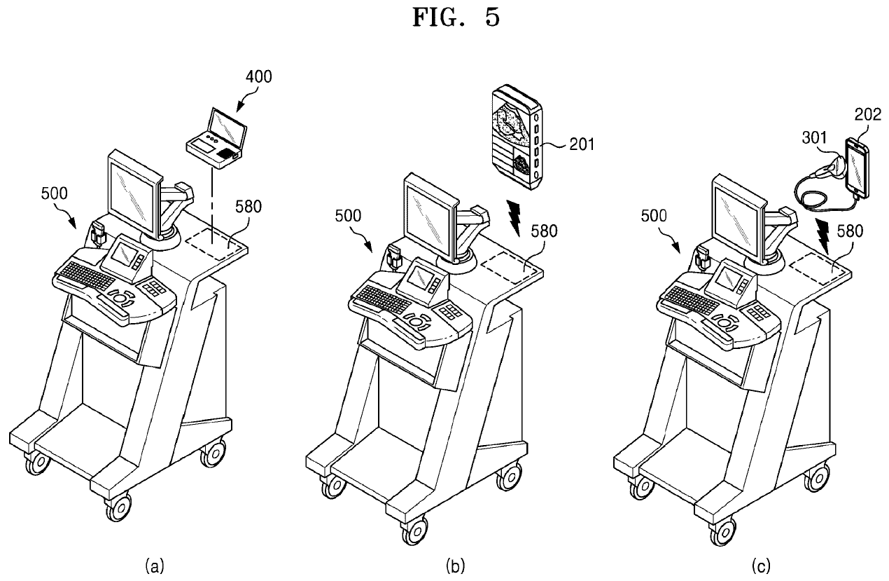

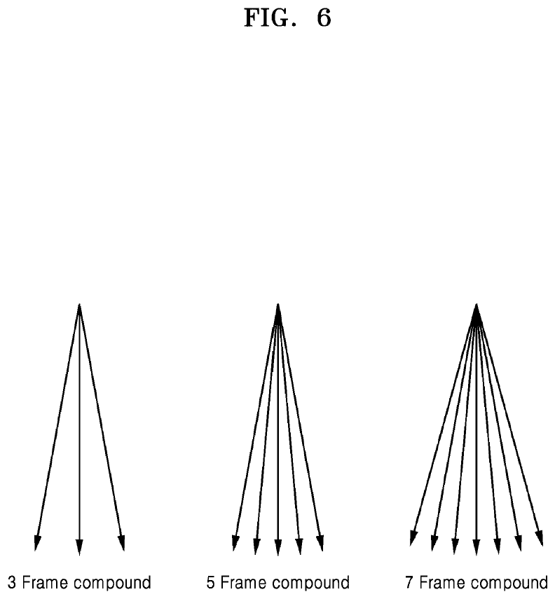

FIG. 6 is a diagram for explaining an ultrasound spatial compound imaging method according to the related art.

In the spatial compound imaging method according to the related art, three or more frames with different steering angles are combined by using transmission Tx with different steering angles, and an image is combined by using a plurality of frame data combined in this way.

FIG. 6 illustrates cases in which the number of frames combined according to the transmission Tx (Tx steer frame) is three, five, and seven, according to the method according to the related art, which essentially includes Tx that is not steered (non-steer Tx) in which the steering angle is 0.

In the present specification, the transmission Tx is disclosed as transmission Tx or Tx, Tx according to a transmission Tx steering angle is disclosed as a non-steer Tx, a right steer Tx, and a left steer Tx, and the transmission Tx frame (Tx steer frame) according thereto is disclosed as a Tx non frame, a Tx right frame, and a Tx left frame.

As illustrated in FIG. 6, according to the related art, as spatial compound is performed by including the Tx non frame that is a frame by Tx that is not steered (Tx non frame) and further including the Tx right frame and the Tx left frame that are frames by the transmission Tx steered to the right and the left, respectively, spatial compound is performed by using at least three Tx steer frames.

In other words, according to the related art, three-frame data by center (non-steer), left steer, and right steer Tx are basically used, and when five-frame data is to be combined, center, left steer1, left steer2, right steer1, and right steer2 are used.

However, when the number of Tx steer frame data used for compound is increased as in the method according to the related art, although there is an advantage of increasing a speckle reduction effect, there are problems such as a phenomenon in which a compound image appears slow when there is a movement of the probe or a target human body during the diagnosis process, a blurring phenomenon in which the sharpness of the compound image deteriorates, and an image dragging phenomenon in the direction of movement.

The present disclosure is intended to improve the problem that occurs due to a large number of frame data used in the spatial compound according to the related art, and to reduce the number of Tx steer frame data used in the spatial compound compared to the related art. Accordingly, the present disclosure has the effect of minimizing the slowness of the image, the degradation of sharpness, and the occurrence of the image dragging phenomenon.

Additionally, the present disclosure is intended to prevent speckle patterns that increase according to the decrease in the number of Tx steer frame data as described above. In other words, in order to maintain the speckle reduction effect, an embodiment of the present disclosure includes a method of combining reception Rx data considering the steering angle of Tx (Tx steer angle).

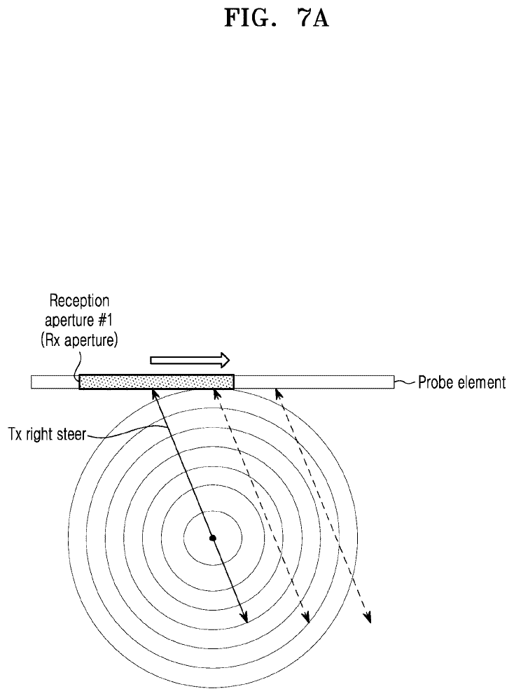

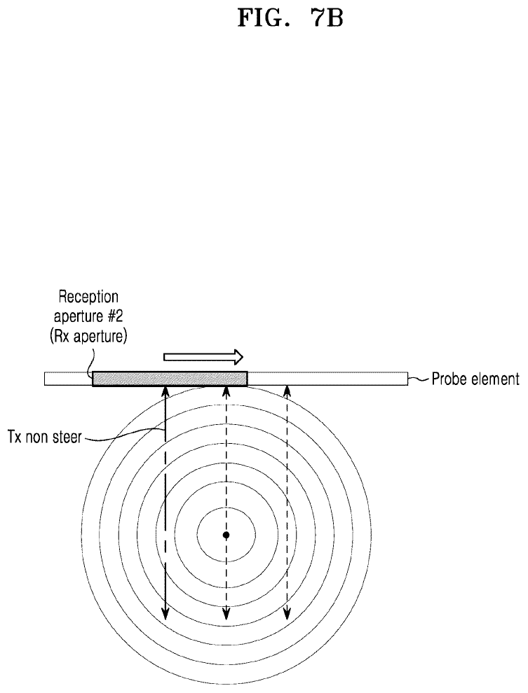

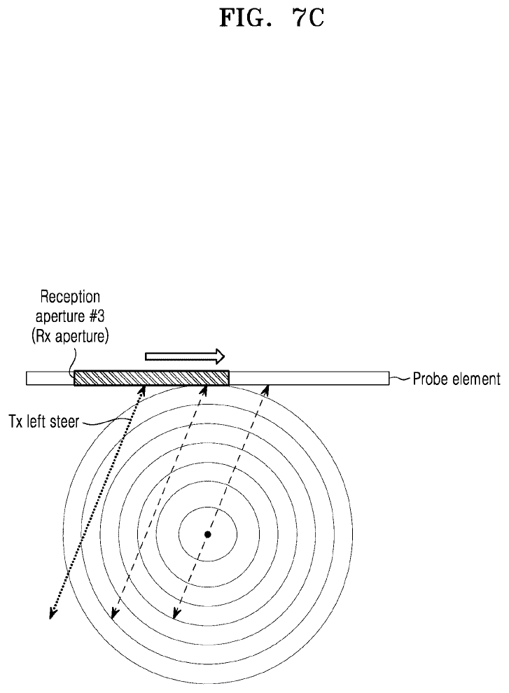

FIGS. 7A to 7D are diagrams for explaining a transceiving method in the ultrasound spatial compound imaging method according to the related art.

FIG. 7A illustrates an image construction method by using the right steer Tx, FIG. 7B illustrates an image construction method by using the non-steer Tx, and FIG. 7C illustrates an image construction method by using the left steer Tx.

First, FIG. 7A illustrates the right steer Tx and illustrates a process of generating frame data by the right steer Tx by repeating transceiving while moving along a scan line, FIG. 7B illustrates, with the non-steer Tx, a process of generating frame data by the non-steer Tx by repeating transceiving while moving along the scan line scan line, and FIG. 7C illustrates, with the left steer Tx, a process of generating frame data by the left steer by repeating transceiving while moving along the scan line scan line scan line. As illustrated in FIGS. 7A to 7D, in the spatial compound imaging method according to the related art, a method in which the center of transceiving is the same is used.

In other words, in the ultrasound spatial compound imaging method according to the related art, as illustrated in FIG. 7D, the weight centers of reception apertures (Rx apertures) 1, 2, and 3 are the same as the transmitting centers thereof, and pieces of data corresponding to the spatially same position are obtained as the Tx steer frame by the right steer Tx, the non-steer Tx, and the left steer Tx, and the respective frames are combined, thereby generating an image.

Unlike the above, the Tx in the ultrasound spatial compound imaging method according to the present disclosure does not include the non-steer Tx that is not steered, and thus an embodiment of the present disclosure is characterized in that it does not include the Tx non frame.

Transmission Tx scan lines in the present disclosure are all set, and a frame according to the transmission Tx (Tx steer frame) may include a frame formed by Tx steered to the right (Tx right frame) and a frame formed by the Tx steered to the left (Tx left frame).

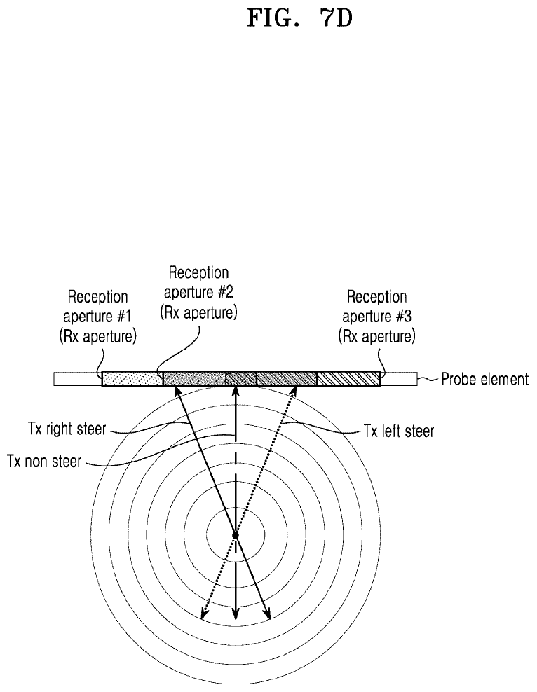

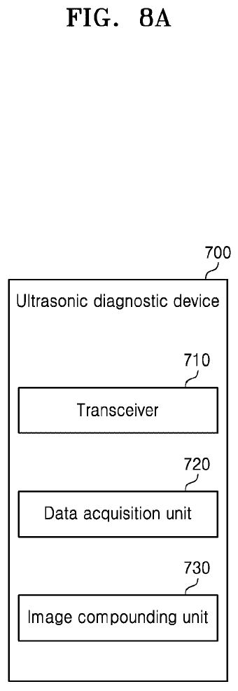

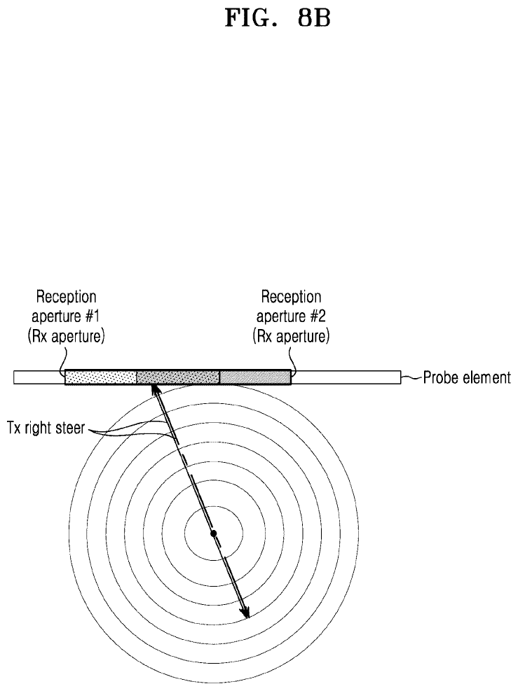

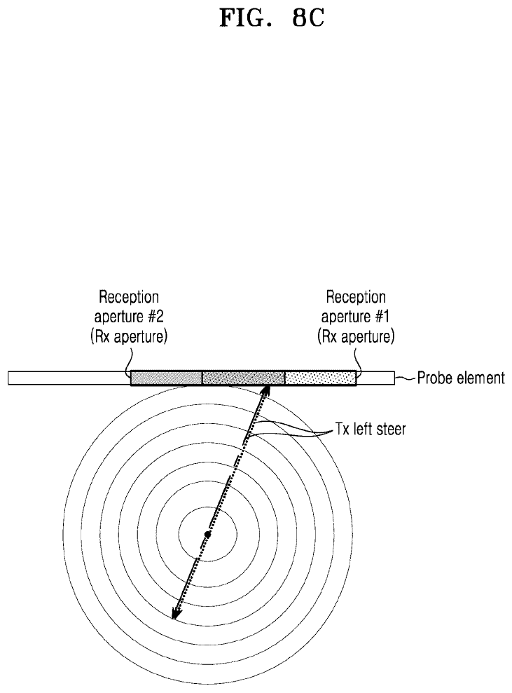

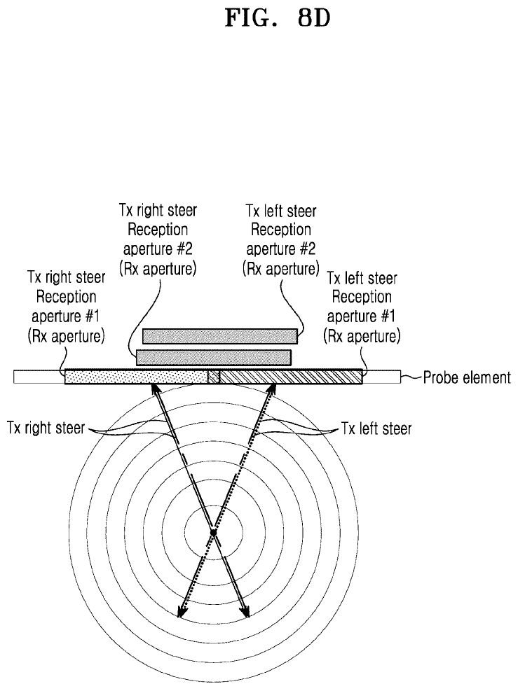

FIGS. 8A to 8D are diagrams for explaining a transceiving method of the ultrasound spatial compound imaging method, in an ultrasonic diagnostic device 700 according to an embodiment of the present disclosure.

As illustrated in FIG. 8A, an ultrasound spatial compound imaging method according to an embodiment of the present disclosure includes a transceiver 710 that transmits an ultrasound signal to an object and receives a reflected ultrasound signal reflected from the object, a data acquisition unit 720 that operates to obtain frame data corresponding to each of a plurality of frames generated based on the transceived signal, and an image compounding unit 730 forming an ultrasound spatial compound image by performing spatial compounding on a plurality of pieces of frame data, wherein the transmission Tx of the present disclosure includes only the Tx steered to the right (right steer Tx) and the Tx steered to the left (left steer Tx).

Accordingly, a plurality of frames (Tx steer frame) according to the transmission Tx may include the frame (Tx right frame) formed by Tx steered to the right and the frame (Tx left frame) formed by the Tx steered to the left.

The frames (Tx steer frame) according to the transmission Tx may be even-numbered, and according to an embodiment, the frames may include pairs of the right steer frame and the left steer frame, excluding the non-steer frame.

In the present disclosure, the number of reception Rx scan lines having different reception Rx settings per transmission Tx may be two or more.

According to an embodiment, the transmission Tx includes a plurality of transmissions Tx, two or more reception Rx scan lines may be obtained from each transmission Tx, and the two or more reception Rx scan lines according to the same transmission Tx may have the same focusing delay.

In the present disclosure, the data acquisition unit 720 may obtain one piece of frame data by using a plurality of reception Rx scan lines generated from the same transmission Tx, and the image compounding unit 730 may form an ultrasound spatial compound image by performing spatial compounding on the plurality of pieces of frame data obtained as above.

FIG. 8B is a diagram for explaining an image construction method by using the right steer Tx according to the present disclosure, which illustrates that two reception apertures (Rx apertures), that is, two Rx scan lines, are obtained for one transmission Tx, wherein the two Rx scan lines according to the same transmission Tx have the same focusing delay. As illustrated in FIG. 8B, in the present disclosure, the respective Rx scan lines may have different positions of reception apertures (Rx apertures) 1 and 2, and different Rx apodization windows.

FIG. 8C is a diagram for explaining an image construction method by using the left steer Tx according to the present disclosure, which illustrates, as in FIG. 8B, two reception apertures (Rx apertures), that is, two Rx scan lines, are used for one transmission Tx. Likewise, the two Rx scan lines according to the same transmission Tx may have the same focusing delay, but have different positions of reception apertures (Rx apertures), and different Rx apodization windows.

FIG. 8D is a diagram for explaining a spatial compound imaging method by using FIGS. 8B and 8C according to the present disclosure, in which the transmission Tx includes the right steer Tx and the left steer Tx, respectively including the reception apertures (Rx apertures) 1 and 2 for the right steer Tx as illustrated in the upper left side of the drawing, and the reception apertures (Rx apertures) 1 and 2 for the left steer Tx as illustrated in the upper right side of the drawing.

In other words, in the present disclosure, an image is constructed by performing spatial compounding on four pieces of data corresponding to the same position, in which the number of Tx steer frames in use is two and subsequently there are one transmission center and two reception centers. Although FIG. 8D illustrates two reception centers, in the present disclosure, the number of reception centers may be two or more.

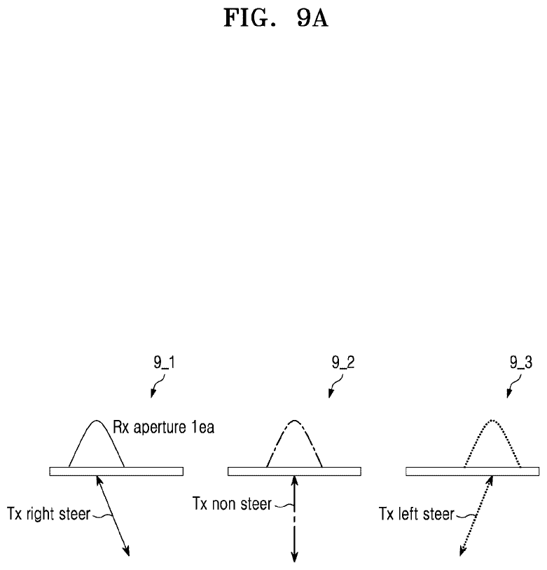

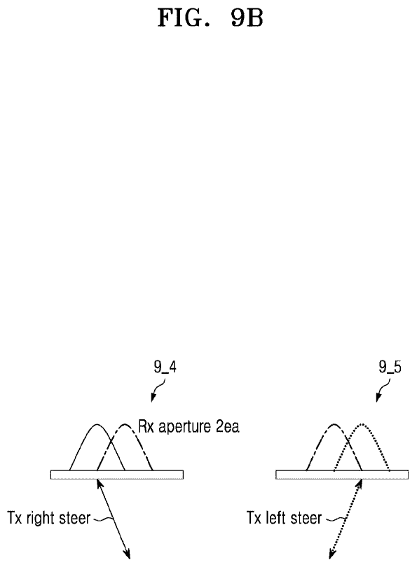

FIGS. 9A and 9B are diagrams for comparing the ultrasound transceiving method according to the related art and the ultrasound transceiving method according to an embodiment of the present disclosure, in the ultrasound spatial compound imaging method.

FIG. 9A illustrates an ultrasound spatial compound imaging method according to the related art, and FIG. 9B illustrates the ultrasound spatial compound imaging method according to the present disclosure.

The method according to the related art illustrated in FIG. 9A includes right steer Tx (9-1) and left steer Tx (9-3) while including non-steer Tx illustrated in (9-2). In each Tx, a single reception aperture (Rx aperture) is used, and three frames (Tx steer frame) are combined by using a total of three pieces of data only.

In contrast, the present disclosure illustrated in FIG. 9B differs from the method according to the related art in that the former include only right steer Tx (9-4) and left steer Tx (9-5) without including the non-steer Tx. In other words, by not using the non-steer Tx, a frame according to the non-steer Tx is not used, and thus the number of frames (Tx steer frame data) used in the compound may be reduced to two. As an effect of the present disclosure, the phenomenon in which a compound image appears slow, the blurring phenomenon, and the image dragging phenomenon may be minimized.

Additionally, in order to minimize the occurrence of a speckle pattern according to the reduction in the number of frames (Tx steer frame data) used in the compound, in the present disclosure, reception data (Rx data) are additionally generated to replace the frame that is not steered (non-steer frame), and thus the speckle reduction effect may be increased.

In detail, as illustrated in FIG. 9B, the present disclosure includes two reception apertures (Rx apertures) for each, and thus two reception Rx scan lines are received in each transmission Tx direction. Accordingly, in the present disclosure, two frames (Tx steer frame) are combined by using a total of four pieces of data.

As a result, in the present disclosure, by reducing the number (two) of frames used in the compound compared with the number (four) according to the related art, the reduced sharpness and the image dragging phenomenon are improved. By increasing the reception aperture (Rx aperture) received in the same transmission Tx direction by two each, the number of pieces of data used in the compound is increased from three (FIG. 9A) according to the related art to four (FIG. 9B). Consequently, the significance lies in maintaining the speckle reduction effect.

FIG. 9B describes that each transmission Tx direction has two reception apertures (Rx apertures), but this is the minimum number, and each may include N reception apertures (Rx apertures).

In this case, the number of pieces of data used in the compound increases more so that the speckle reduction effect is improved further.

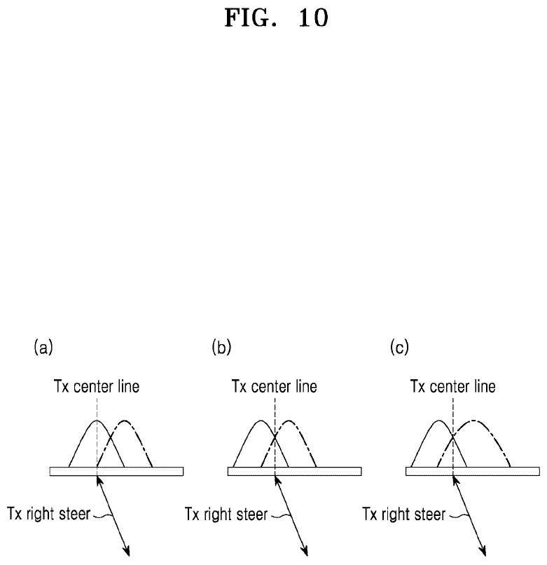

FIG. 10 is a diagram for explaining a position of a reception aperture in the ultrasound spatial compound imaging method according to an embodiment of the present disclosure.

In the present disclosure, a plurality of reception apertures (Rx apertures) may differ in at least one of a size, a position, and an apodization window.

FIG. 10 illustrates an example of using a linear probe in an ultrasound spatial compound image process according to the present disclosure, in which (a) is an embodiment in which the center line of the transmission Tx matches the center of one reception aperture (Rx aperture), and (b) is an embodiment in which the center line of the transmission Tx matches the intersection of two reception apertures (Rx apertures), and (c) is an embodiment in which the center line of the transmission Tx matches the intersection of two reception apertures (Rx apertures), but unlike (b), the position and size of the reception aperture (Rx aperture) located on the right are different from those on the right.

As illustrated in FIG. 10, the positions of the two reception apertures (Rx apertures) may be configured in various combinations with respect to the center line of the transmission Tx, and the sizes of the two reception apertures (Rx apertures) may be different from each other.

The present disclosure is significant in that, by adjusting the spacing between two reception apertures (Rx apertures) according to the steering angle of Tx (Tx steer angle), it is possible to maintain the same effect as including a frame by the non-steer Tx (non-steer Tx frame), with only the right steer Tx and the left steer Tx, without including the non-steer Tx.

In other words, the present disclosure shows that, by combining the position and size of the reception aperture (Rx aperture), the speckle reduction effect may be maintained even without the non-steer Tx frame (non-steer frame).

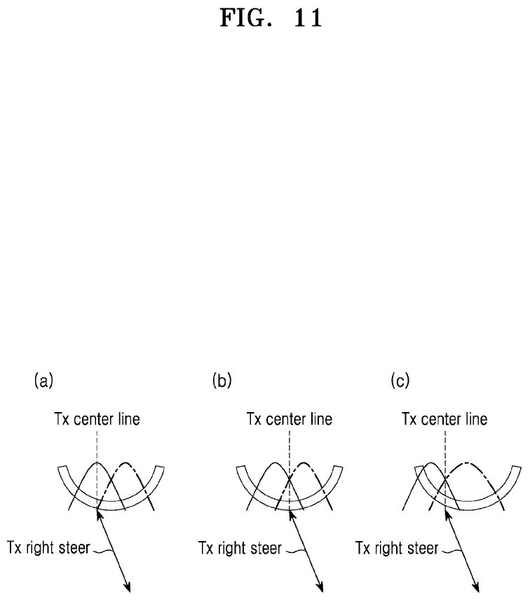

FIG. 11 is a diagram for explaining a position of a reception aperture in an ultrasound spatial compound imaging method according to another embodiment of the present disclosure.

Unlike FIG. 10, FIG. 11 illustrates an example of using a curved probe, in which even when the Tx steer angle is the same, a combination of reception apertures (Rx apertures) is adjusted according to the shape of a probe.

Like FIG. 10, in (a), (b), and (c) of FIG. 11, (a) is an embodiment in which the center line of the transmission Tx matches the center of one reception aperture (Rx aperture), and (b) and (c) are embodiments in which the center line of the transmission Tx matches the intersection of two reception apertures (Rx apertures), but the position and size of the reception aperture (Rx aperture) located on the right are different from those on the right.

As described in FIGS. 10 and 11, in the ultrasound spatial compound imaging method according to the present disclosure, the size or spacing of the reception aperture (Rx aperture) may be adjusted according to the size of Tx steer angle and the shape of probe, and as such, by adjusting the combination of reception aperture (Rx aperture), the speckle reduction effect may be maintained with only the Tx right steer and the Tx left steer.

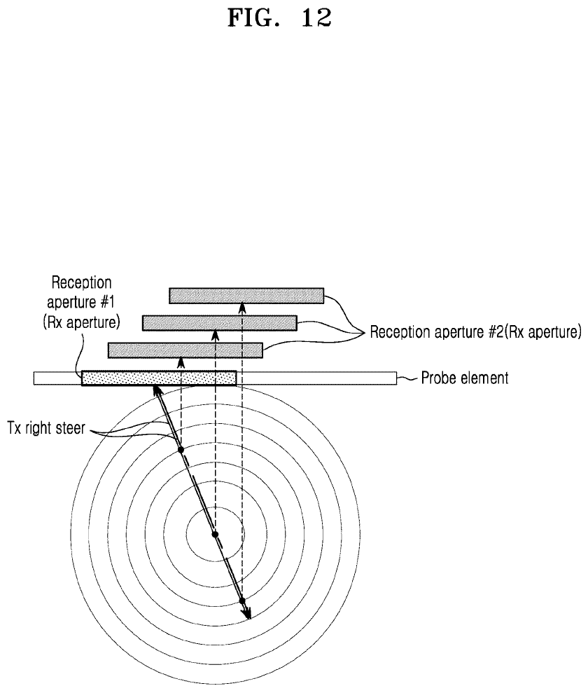

FIG. 12 is a diagram for explaining a position of a reception aperture position in an ultrasound spatial compound imaging method according to another embodiment of the present disclosure.

According to the present disclosure, the weight centers of a plurality of reception apertures (Rx apertures) may move for each depth, and according to an embodiment, the movements of the plurality of reception apertures (Rx apertures) may be independent.

FIG. 12 is a diagram illustrating an example of an image construction method according to the Tx right steer. As illustrated, the weight center of a reception aperture (Rx aperture) may be moved for each depth. Although it is illustrated in the drawing that only reception aperture (Rx aperture) #2 moves, it is possible that reception aperture (Rx aperture) #1 also moves.

As the depth increases, the weight center of reception aperture (Rx aperture) in the present disclosure may move laterally to be perpendicular to a target position.

When two pieces of data having different reception Rx settings are obtained from a Tx steer frame, one of the two pieces of data needs to replace data obtained under the non-steer Tx condition, and thus, in order to be most similar to the reception data (Rx data) in the non-steer Tx that is perpendicular to the surface of the probe, the reception aperture (Rx aperture) needs to move in a direction perpendicular to the surface of the probe.

According to an embodiment, in the process of obtaining one piece of frame data by using a plurality of reception Rx scan lines generated from the same transmission Tx, a weight may be applied to each reception Rx scan lines.

In detail, signals of the plurality of Rx scan lines may be combined by applying a weight for each reception Rx scan line, and the weight may vary depending on the depth.

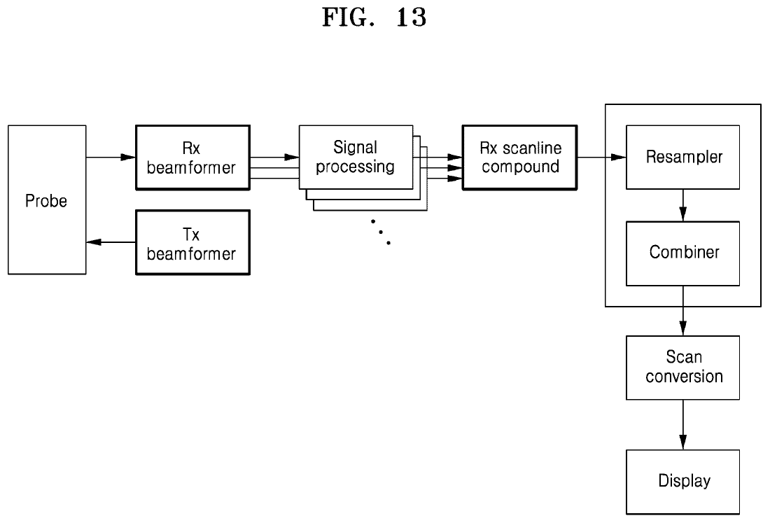

FIG. 13 is a diagram for explaining an ultrasound spatial compound imaging method according to an embodiment of the present disclosure.

An embodiment of the present disclosure is described with reference to the configuration illustrated in FIG. 13.

First, a Tx beamformer may support even-numbered Tx steer frames including a right steer frame and a left steer frame, excluding a non-steer frame in a spatial compound mode.

An Rx beamformer may output N beamforming results having different sizes, positions, or Rx apodization window settings of reception apertures (Rx apertures) according to the Tx steer angle.

A signal processing unit (Signal Processing) performs functions from the beamforming output data to envelop detection, may process N beamforming results simultaneously according to an embodiment, and may perform a method of performing N times with one logic a process from the beamforming to the signal processing process or only the signal processing process, by using a memory.

Next, in a reception Rx scan line compounding unit (Rx scan line compound), data of the reception Rx scan lines may be combined.

In the present disclosure, the pieces of data of a plurality of reception Rx scan lines generated from the same transmission Tx have the same spatial position so as to be combined immediately, and during compounding, signals of the plurality of reception Rx scan lines may be combined after applying a weight value to each reception Rx scan line.

For example, assuming that there are pieces of data A and B of the reception Rx scan line generated from the left steer Tx, A and B are summed by applying a weight thereto and finally only one piece of data is output, a resampler or combine function for the transmission Tx spatial compound function according to the related art may be identically used.

In an example, assuming that there are two reception apertures (Rx apertures) by the right steer Tx and the reception Rx scan line data according thereto are A and B, and that there are two reception apertures (Rx apertures) by the left steer Tx and the reception Rx scan line data according thereto are C and D, the reception Rx scan line data by the right steer Tx may be gain1×A+gain2×B, and the reception Rx scan line data by the left steer Tx may be gain3×C+gain4×D.

The weight value for each of the scan line data A, B, C, and D may be in the form of sequentially 1, 1, 1, and 1, or 1, 0.5, 0.5, and 1, but the disclosure is not limited thereto.

Also, according to another embodiment, the weight values for A, B, C, and D may be set differently depending on the depth.

In the ultrasound spatial compound imaging method according to the embodiment of the present disclosure, a scan line of the transmission Tx forming the frame formed by Tx steered to the right (Tx right frame) and a scan line of the transmission Tx forming the frame formed by the Tx steered to the left (Tx left frame) may each obtain two or more reception Rx scan lines, and the weight centers of reception apertures (Rx apertures) of a plurality of reception Rx scan lines may be different from each other.

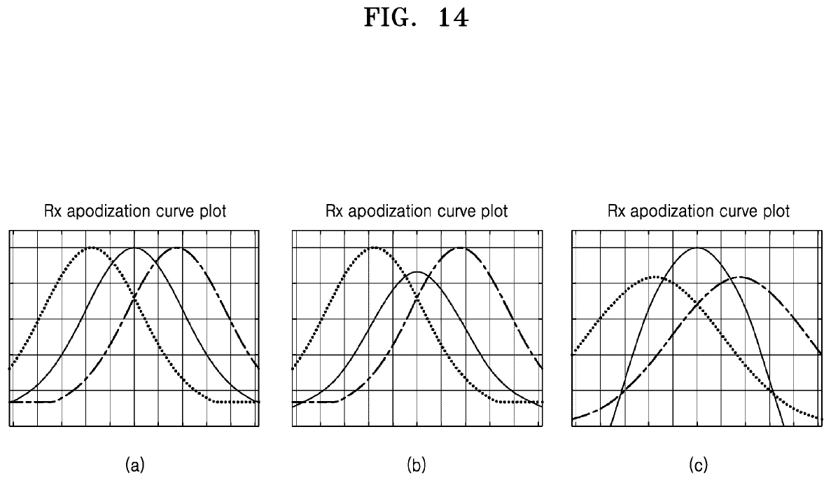

FIG. 14 is a diagram for explaining a case in which Rx apodization windows are different from one another, in an ultrasound spatial compound imaging method according to another embodiment of the present disclosure.

First, (a), (b), and (c) of FIG. 14 all have the same size of a reception aperture (Rx aperture).

In FIG. 14, (a) illustrates a case in which the centers of apodization windows are different from one another while gains of the apodization windows are the same, (b) illustrates a case in which the centers and gains of apodization windows are all different from one another, and (c) illustrates a case in which the centers, widths, and gains of apodization windows are all different from one another.

Generally, scattering is a phenomenon in which ultrasonic signal spreads in all directions regardless of an incident angle, when the position of the Rx aperture and the apodization window are similar to each other, speckle patterns appears similar regardless of the Tx steer angle. In other words, when transmission Rx settings are the same in spite of different Tx steer angles, speckle patterns appear similar to each other.

Through FIG. 14, it may be seen that the same result can be obtained while the sizes and positions of reception apertures (Rx apertures) are the same and the apodization windows are different from each other.

When the reception apertures (Rx apertures) are the same and the Rx apodization windows are changed, the weight centers of the reception apertures (Rx apertures) are changed so that the same effect as moving the position of the reception aperture (Rx aperture) may be obtained.

In this state, the weight value may be additionally adjusted to compensate for signal reduction according to the probe directivity angle in the Rx apodization window.

In FIG. 14, (a) illustrates a case where there is no additional weight, (b) illustrates a case of applying sensitivity decrease compensation according to the probe directivity angle, in which additional gains are applied to windows located on both sides because generally the direction perpendicular to the surface of a probe has the best sensitivity, and (c) illustrates a case in which, although the same method as the method in (b) is employed, the area of the Rx apodization window has a large difference, indicating that the peak value of the center window is larger.

Additionally, an experiment through a phantom was performed for comparison between 3 spatial compound imaging (SCI) that is a method of combining the three Tx steer frames (using Tx right steer, Tx non-steer, and Tx left steer) according to the related art with 2 spatial compound imaging (SCI) that is a method of combining two Tx steer frames (using Tx right steer and Tx left steer only) according to the present disclosure, and is described below.

While both methods have the same Tx steer angle settings, in the 3 spatial compound imaging (SCI), the same Rx apodization window was used regardless of the Tx steer angle, and in the 2 spatial compound imaging (SCI), different Rx apodization windows were used according to the Tx steer angles. The weights of the reception Rx scan line spatial compound were 1.0, 0.5, 0.5, and 1.0.

TABLE 1

| | | Rx | Axial Resolution | | | Lateral Resolution | | | Mean | | |
|---|---|---|---|---|---|---|---|---|---|---|---|
| | Tx | Data | [mm] | | | [mm] | | | Speckle | | |
| | Frame | Compound | Target | | | | | | Size | | |
| Classification | Number | Number | 1 | 2 | 3 | 1 | 2 | 3 | [mm²] | Mean | Variance |
| 3 SCI | 3 | 3 | 1.09 | 1.54 | 1.38 | 1.16 | 1.83 | 2.18 | 2.11 | 67.53 | 17.34 |
| 2 SCI | 2 | 4 | 1.10 | 1.52 | 1.41 | 1.23 | 1.74 | 2.21 | 2.18 | 68.01 | 15.21 |

As shown in the experimental results in [Table 1] above, there is almost no difference in the space resolution (axial/lateral resolution) according to the present disclosure of the 2 spatial compound imaging (SCI).

However, in the result according to the present disclosure of the 2 spatial compound imaging (SCI), the speckle pattern variance is low, and it may be confirmed that the speckle reduction effect is higher in the present disclosure.

This is because, in the present disclosure, the center position of the reception aperture (Rx aperture) is divided left and right with the center position of the transmitting aperture (Tx aperture) as a reference (see FIG. 9B), and the speckle reduction effect is improved because the difference in the speckle pattern becomes larger.

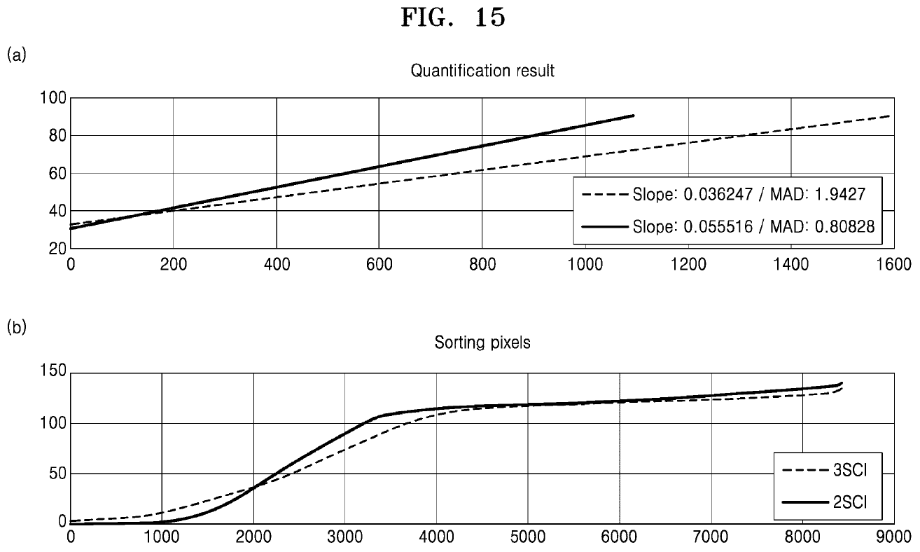

FIG. 15 is a diagram for explaining effects of the ultrasound spatial compound imaging method of the present disclosure.

FIG. 15 shows a result of a comparative experiment to check a degree of blurring due to motion in an image according to the ultrasound spatial compound imaging method of the present disclosure.

The experiment was performed under conditions of a frame rate of 24 Hz and a steering angle of 8 degrees for each of right and left for each of a case by the method according to the related art (3 spatial compound imaging (SCI)) and a case according to the present disclosure (2 spatial compound imaging (SCI)).

The greater the slope of the graph in FIG. 15 according to the experimental results, the less a degree of blurring compared to the motion, and it may be confirmed that the slope of the graph corresponding to the 2 spatial compound imaging (SCI) of the present disclosure is greater than that of the 3 spatial compound imaging (SCI) corresponding to the method according to the related art.

In other words, the image to which the 2 spatial compound imaging (SCI) is applied has a less degree of blurring due to motion and shows motion faster than the image to which the 3 spatial compound imaging (SCI) according to the related art is applied.

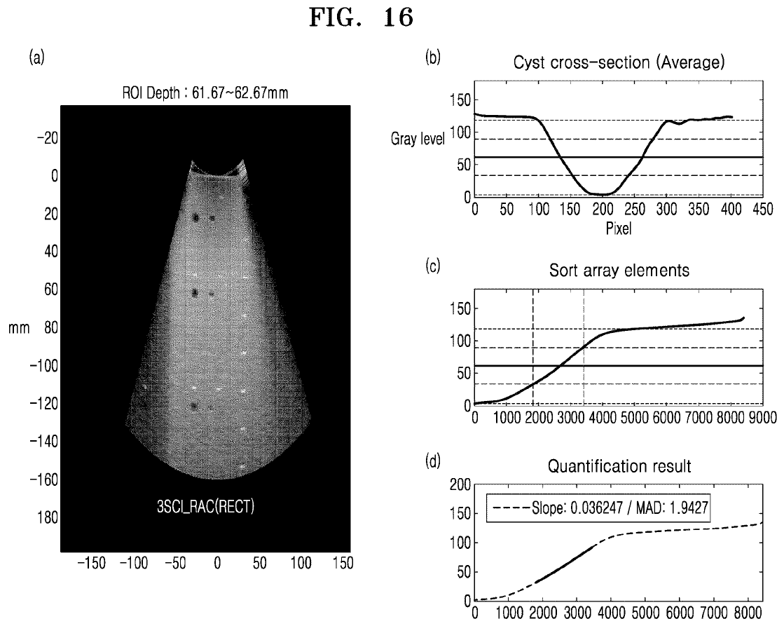

FIG. 16 is a diagram for explaining effects of the ultrasound spatial compound imaging method according to the related art.

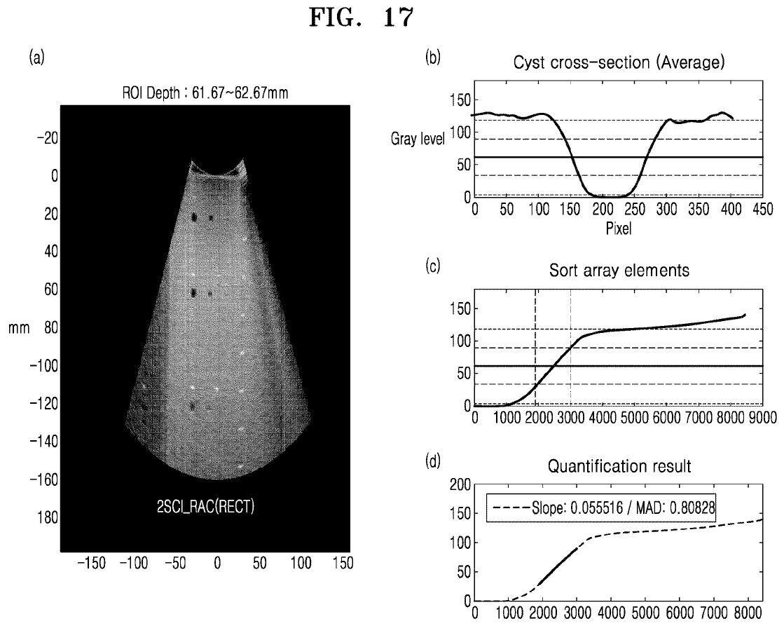

FIG. 17 is a diagram for explaining effects of the ultrasound spatial compound imaging method according to the present disclosure.

FIG. 16 shows an experiment about a degree of blurring due to motion by the 3 spatial compound imaging (SCI), and FIG. 17 shows an experiment about a degree of blurring due to motion by the 2 spatial compound imaging (SCI).

Generally, when acquiring a phantom image by moving a probe at a constant speed using a 1-axis motor, the boundary of a cyst target has a gentle slope value due to blurring caused by motion, and when in a stationary state with no motion, the boundary has a shape close to a right angle.

FIGS. 16 and 17 show a comparison of the quantitative experimental results of a degree of blurring. It may be confirmed that the image obtained by combining 2 sheets of frames shows the boundary of the cyst target better than the image obtained by combining 3 sheets of Tx frames (FIG. 16), and that the blurring due to motion occurs less.

In other words, it may be confirmed that the sharpness shown in the ultrasound image on the left in each drawing is clearer in FIG. 17, and it may be confirmed from the quantification experiment result on the lower right that the slope of the graph is larger in FIG. 17 than in FIG. 16 (0.036247<0.055516), which shows that the method of FIG. 17 according to the present disclosure has the effect of reducing the degree of blurring due to motion and making motion appear faster.

The ultrasound spatial compound imaging method according to the ultrasound diagnostic method according to an embodiment of the present disclosure includes transmitting, by the transceiver 710, an ultrasound signal to an object and receiving, by the transceiver 710, a reflected ultrasound signal reflected from the object, operating the data acquisition unit 720 to obtain frame data corresponding to each of a plurality of frames generated based on the transceived signal, and forming an ultrasound spatial compound image by performing spatial compounding, by the image compounding unit 730, on a plurality of pieces of frame data, wherein transmission Tx includes Tx steered to the right (Tx right steer) and Tx steered to the left (Tx left steer) only.

As detailed contents related to the respective operations are described in association with the ultrasound device 700 according to the embodiment of the present disclosure, detailed descriptions thereof are omitted.

As presented above, the disclosed embodiments are described with reference to the accompanying drawings. It will be understood by those skilled in the art that various modifications and variations can be made in the present disclosure without departing from the spirit or scope of the disclosure. The embodiments disclosed herein are merely illustrative and are not intended to limit the scope of the present disclosure.

1. An ultrasonic diagnostic device comprising:
a transceiver configured to transmit an ultrasound signal to an object and receive a reflected ultrasound signal reflected from the object;
a data acquisition unit configured to operate to obtain frame data corresponding to each of a plurality of frames generated based on the transceived signal; and
an image compounding unit configured to form an ultrasound spatial compound image by performing spatial compounding on the plurality of pieces of frame data,
wherein transmission (Tx) includes Tx steered to right (right steer Tx) and Tx steered to left (left steer Tx) only.

2. The ultrasonic diagnostic device of claim 1, wherein
a plurality of frames (Tx steer frames) according to the transmission (Tx) do not include a frame by Tx that is not steered (non-steer frame), and include a frame formed by Tx steered to the right (Tx right frame) and a frame formed by Tx steered to the left (Tx left frame).

3. The ultrasonic diagnostic device of claim 1, wherein
frames (Tx steer frames) according to the transmission (Tx) are even-numbered.

4. The ultrasonic diagnostic device of claim 1, wherein
a number of reception (Rx) scan lines having different reception (Rx) per transmission (Tx) settings is two or more.

5. The ultrasonic diagnostic device of claim 1, wherein
the transmission (Tx) includes a plurality of transmissions, and two or more reception (Rx) scan lines are obtained from each transmission (Tx).

6. The ultrasonic diagnostic device of claim 5, wherein
the two or more reception (Rx) scan lines according to a same transmission (Tx) have a same focusing delay.

7. The ultrasonic diagnostic device of claim 4, wherein
a plurality of reception apertures (Rx apertures) differ in at least one of a size, a position, and an apodization window.

8. The ultrasonic diagnostic device of claim 4, wherein
weight centers of a plurality of reception apertures (Rx apertures) move for each depth.

9. The ultrasonic diagnostic device of claim 4, wherein
a plurality of reception apertures (Rx apertures) move independently.

10. The ultrasonic diagnostic device of claim 4, wherein the data acquisition unit is further configured to obtain one piece of frame data by using the plurality of reception (Rx) scan lines generated from a same transmission (Tx).

11. The ultrasonic diagnostic device of claim 10, wherein
compounding signals of a plurality of reception (Rx) scan lines is performed by applying respective weights to signals of the plurality of reception (Rx) scan lines.

12. The ultrasonic diagnostic device of claim 11, wherein
the weights differ depending on depth.

13. The ultrasonic diagnostic device of claim 2, wherein
a scan line of the transmission (Tx) that forms the frame formed by the Tx steered to the right (Tx right frame) and a scan line of the transmission (Tx) that forms the frame formed by the Tx steered to the left (Tx left frame) each obtain two or more reception (Rx) scan lines.

14. The ultrasonic diagnostic device of claim 13, wherein
weight centers of reception apertures (Rx apertures) of the plurality of reception (Rx) scan lines are different from each other.

15. An ultrasound diagnostic method comprising:
transmitting, by a transceiver, an ultrasound signal to an object and receiving, by the transceiver, a reflected ultrasound signal reflected from the object;
operating a data acquisition unit to obtain frame data corresponding to each of a plurality of frames generated based on the transceived signal; and
forming, by an image compounding unit, an ultrasound spatial compound image by performing spatial compounding on a plurality of pieces of frame data,
wherein transmission (Tx) includes Tx steered to right (right steer Tx) and Tx steered to left (left steer Tx) only.
